(19) United States
(12) Patent Application Publication
(10) Pub. No.: US 2026/0325096 A1
(43) Pub. Date: Oct. 22, 2026

(54) IMAGING APPARATUS, METHOD FOR TIME-RESOLVED MEDICAL IMAGING, AND COMPUTER-READABLE MEDIUM

(71) Applicant: Siemens Healthineers AG, Forchheim (DE)

(72) Inventors: Annette SCHWARZ, Erlangen (DE); Jannis DICKMANN, Erlangen (DE); Fernando VEGA, Erlangen (DE); Simon SCHMIDT, Nuernberg (DE)

(73) Assignee: Siemens Healthineers AG, Forchheim (DE)

(21) Appl. No.: 19/468,129

(22) Filed: Feb. 3, 2026

(30) Foreign Application Priority Data
Feb. 4, 2025 (EP) 25155786.4

(51) Int. Cl. A61B 6/00; A61B 6/40

(52) U.S. Cl. CPC A61B 6/5258; A61B 6/4035; A61B 6/5211

(57) ABSTRACT

One or more example embodiments relates to an imaging apparatus including a data-acquisition module configured to capture and receive a plurality of temporally ordered datasets used in an image generation process for an entity in at least one predefined time period, wherein the plurality of temporally ordered datasets comprise spatial domain information and time domain information; at least one denoising module coupled to the data-acquisition module and comprising a settable/adjustable bilateral filter, wherein the at least one denoising module is configured to set/adjust filter parameters of the settable/adjustable bilateral filter using the received spatial domain information and the time domain information, such to denoise the plurality of temporally ordered datasets using the settable/adjustable bilateral filter and to obtain a plurality of denoised temporally ordered datasets; and an outputting module configured to output at least one time-resolved medical image for the entity using the plurality of denoised temporally ordered datasets.

### CROSS-REFERENCE TO RELATED APPLICATION(S)

The present application claims priority under 35 U.S.C. § 119 to European Patent Application No. 25155786.4, filed Feb. 4, 2025, the entire contents of which is incorporated herein by reference.

### FIELD

One or more example embodiments relates to an imaging apparatus. One or more example embodiments further relates to a method for time-resolved medical imaging employing in the imaging apparatus, a storage medium and a computer program product.

### RELATED ART

The clinical use of the medical imaging is of essential importance in the human medicine and veterinary medicine. From two-dimensional, 2D, medical imaging, which offers flat, linear views, limited to length and width to three-dimensional, 3D, medical imaging, which adds the third dimension of depth, the effort to increasing the dimensions of medical image to provide a more comprehensive medical imaging presentation has never ceased.

In recent years, time-resolved medical imaging provides a more advanced form of imaging. The time-resolved medical imaging is a medical imaging technology that considers the time as the additional dimension and aims to represent the motion of the regions of interest over time. Through time-resolved medical imaging, the movement of the regions of interest can be better observed.

In addition, when an ionizing radiation is involved in the medical imaging process, the higher the radiation dose contributed to the image, the less image noise will be apparent and the easier the low-contrast structures will be perceived. However, the possible damage done to the human or animal bodies is proportional to the absorbed dose of the ionizing radiation to the organs of the bodies. Since the time-resolved medical imaging usually involves capturing multiple static medical images over predefined time points, the number of repeat scans a patient or an animal can receive for continuously adapting the radiotherapy treatment plan might be limited.

Therefore, the experts have been striving to improve the imaging apparatus in time-resolved medical imaging to limit the radiation dose used to obtain high-quality medical images, and to provide an improved view of the results of the movement of the regions of interest.

Previous solutions to denoise time-resolved imaging typically involve applying 2D or 3D spatial imaging denoising methods to the time-resolved medical imaging, in which the time domain information in time-resolved medical imaging is not involved in the process of image denoising. In the meantime, since the bilateral filters are successful in smoothing images while preserving edges in images, 2D or 3D spatial imaging denoising methods frequently adopt bilateral filters to filter the image noises.

In a scientific publication, A. M. Mendrik et. al, “TIPS bilateral noise reduction in 4D CT perfusion scans produces high-quality cerebral blood flow maps,” Phys. Med. Biol. 56, 3857-3872 (2011), the use of four-dimensional, 4D, bilateral filters for denoising the medical images of brains scans is explored, which includes a measure of voxel similarity in time-intensity profile besides the 3D spatial domain voxel values. However, fine-tuning the 4D bilateral filters to the specific noise characteristics can be a challenge. Besides, as illustrated in another scientific publication, Wagner, Fabian, et al. “Ultralow-parameter denoising: trainable bilateral filter layers in computed tomography.” Medical physics 49.8, 5107-5120 (2022), the bilateral filters have achieved strong denoising performance for 3D spatial image data and reduced computation load at the same time. However, the time-resolved image data has not been used to train the bilateral filters in this literature. Most image generation or denoising algorithms are built based on deep artificial neural networks with multiple layers and contain hundreds of thousands of trainable parameters, which means that the training process for the denoising algorithm requires intensive computational load. Although training with deep artificial neural networks facilitates extracting complex features from data, it is impossible to fully comprehend the data processing and it has a possibility to produce hallucinations.

### SUMMARY

One or more example embodiments provides an imaging apparatus which implements a novel method for time-resolved medical imaging.

### BRIEF DESCRIPTION OF THE DRAWINGS

Example embodiments are described in greater detail in the following on the basis of the embodiments shown in the schematic figures of the drawings, in which:

FIG. 1 illustrates a block diagram of an example of the image apparatus;

FIG. 2 illustrates a block diagram of another example of the image apparatus;

FIG. 3 illustrates a diagram of an example of a plurality of temporally ordered datasets captured within a time period;

FIG. 4 illustrates a schematic diagram of an example of a filtering process for an exemplar image using a settable/adjustable bilateral filter; and

FIG. 5 illustrates a chart of an example of a method for time-resolved medical imaging.

The appended drawings are intended to provide further understanding of the embodiments of the invention. They illustrate embodiments. Other embodiments and many of the advantages mentioned become apparent in view of the drawings. The elements in the drawings are not necessarily shown to scale.

### DETAILED DESCRIPTION

An imaging apparatus, the imaging apparatus comprising: a data-acquisition module configured to capture and receive a plurality of temporally ordered datasets used in an image generation process for an entity in at least one predefined time period, wherein the plurality of temporally ordered datasets comprise spatial domain information and time domain information; at least one denoising module coupled to the data-acquisition module and comprising a settable/adjustable bilateral filter, wherein the at least one denoising module is configured to set/adjust filter parameters of the settable/adjustable bilateral filter using the received spatial domain information and the time domain information, such to denoise the plurality of temporally ordered datasets using the settable/adjustable bilateral filter and to obtain a plurality of denoised temporally ordered datasets; and an outputting module configured to output at least one time-resolved medical image for the entity using the plurality of denoised temporally ordered datasets.

A method for time-resolved medical imaging, in particular by employing an imaging apparatus according to one or more example embodiments. The method comprising the steps of: capturing and receiving a plurality of temporally ordered datasets used in an image generation process for an entity in at least one predefined time period, wherein the plurality of temporally ordered datasets comprise spatial domain information and time domain information; setting/adjusting filter parameters of a settable/adjustable bilateral filter using the received spatial domain information and the time domain information, such to denoise the plurality of temporally ordered datasets using the settable/adjustable bilateral filter and to obtain a plurality of denoised temporally ordered datasets; and outputting at least one time-resolved medical image for the entity using the plurality of denoised temporally ordered datasets.

A computer program product including a computer program, when program sections of the computer program are executed by a computer, cause the imaging apparatus to execute the method according to one or more example embodiments.

A non-transitory computer-readable storage medium storing thereon the computer program according to one or more example embodiments by a computer.

The proposed method according to one or more example embodiments focuses a novel way to denoise the medical images by employing a settable/adjustable bilateral filter using the spatial domain information and time domain information of the data used in an image generation process. A settable/adjustable bilateral filter is a bilateral filter that may automatically set or adjust its filter parameters and configurations and is thus optimizable. With the adoption of the novel method, when capturing the data used in an image generation process is involving an ionizing radiation or long acquisition time, an amount of deposited radiation dose that necessary to acquire a medical image is reduced without compromising the image quality, such as contrast, temporal resolution and geometric accuracy. The current application aims to combine the benefits of settable/adjustable bilateral filters and the adoption of time domain information of the data used in an image generation process, in order to achieve significant improved medical images.

One or more example embodiments is based on the idea to provide an imaging apparatus which implements a novel method for time-resolved medical imaging. The method focuses on proposing a novel way to denoise the medical images with a settable/adjustable bilateral filter using the spatial domain information and time domain information of the data used in an image generation process. In one or more example embodiments, in order to provide a clearer explanation, the process from the input of data used in an image generation process for an entity to the output of the time-resolved image can be assumed to be a processing pipeline having an input, an output, and a channel between the input and the output.

The input of the processing pipeline is a plurality of temporally ordered datasets used in an image generation process for an entity captured and received in at least one predefined time period. Therefore, the plurality of temporally ordered datasets comprise both the spatial domain information and the time domain information. The entity may refer to the regions of interest of a patient, a sick animal, a healthy person or animal providing comparative data through medical imaging for research or other purposes. The entity may also refer to the non-living objects, such as imaging phantoms that designed to mimic human or animal anatomy or tissue properties.

The processing of the plurality of temporally ordered datasets are involving one or multiple settable/adjustable bilateral filter in the channel of the processing pipeline. The settable/adjustable bilateral filter is setting/adjusting by a denoising module of the imaging apparatus using the spatial domain information and the time domain information of the plurality of temporally ordered datasets. During the setting/adjusting of the settable/adjustable bilateral filter, the spatial domain information and the time domain information of the plurality of temporally ordered datasets are used. The spatial domain information typically involves two or three spatial parameters and one intensity range parameter for the settable/adjustable bilateral filter. The time domain information typically involves the time-intensity profile of the parameters involved in those parameters. In time-resolved medical imaging, each scan generates the image of regions of interest containing the same anatomy in different predefined time points. The different predefined time points may be different time points in the same time period or different time points in the different time periods. The time periods may be time cycles. These scans may slightly deform by the motion of the patients. If the pixel or voxel value differs too much from the surrounding pixel or voxel value from the same predefined time point recording at a different time cycles, it can be assumed that it is noise. The pixel or voxel value can be also assumed that it is noise, when these pixel or voxel value differs too much from the surrounding pixel or voxel value from the scans that contains similar anatomy but are recoded at different predefined time points within one time period or time cycle. Therefore, the more scans perform on the regions of interest, the more facilitative the noise filtering.

The output of the processing pipeline is the time-resolved medical images. The outputting module outputs at least one time-resolved image for the entity using the plurality of denoised temporally ordered datasets.

The imaging apparatus, which employs a novel method for time-resolved medical imaging improves the signal to noise ratio, SNR, of the reconstructed medical images for better representation of lesions in low contrast organs. It allows for dose reduction in time-resolved medical imaging when an ionizing radiation is involving in the imaging process. It allows for acquisition time reduction when a prolonging acquisition time is involving in the imaging process. It further avoids tube cooling conflicts in time-resolved tomographic imaging acquisitions.

Advantageous configurations and developments emerge from the further dependent claims and from the description with reference to the figures of the drawings.

In a possible embodiment of the imaging apparatus, the data-acquisition module is configured to capture and receive the plurality of temporally ordered datasets comprising raw detector data described by a plurality of sinograms. Sinogram is used as a 2D transitional image between the raw detector data and the final medical image in some of the medical imaging processes. The proposed imaging apparatus allows a direct filter action on the sinogram. Taking an example of a region of interest imaged by the computed tomography, CT, scanner, sinogram is generated by transforming the raw X-ray projection data taken from various angles into a 2D graphical representation. The X-ray projection refers to a process that the X-ray tube emits an X-ray beam at a given gantry angle, then the beam passes through the region of interest and is intercepted by an imaging detector. The detector converts the intercepted X-ray beam signal into an electronic signal. The trajectory of the X-ray projection corresponding to the region of interest is following a sine wave, which the X-ray angle and the detector position are taken as the axes of the sinogram. In addition, taking an example of a region of interest imaged with a ring-style positron emission tomography scanner, PET scanner, a region of interest will be injected with a PET agent that will localize the region of interest and decay with the emission of a positron. The positron is then annihilated with an electron, which is leading to a coincidence event detection that their masses are converted to energy in the form of two photons being emitted back-to-back. The annihilation has occurred along the line connecting the two detectors, which is referred to as the line of response, LOR. For collecting the raw detector data, the LOR is characterized by the angle of the orientation of the LOR and the shortest distance between the LOR and the center of the PET gantry. If a large number of LORs are plotted from the same region of interest, the resulting graph is represented by a sine wave. This graph visualizes the raw detector data and is referred to as a sinogram. The proposed imaging apparatus can conduct filter function directly on the sinograms, which facilitates the later data processing step with less noises.

In a possible embodiment of the imaging apparatus, the data-acquisition module is configured to capture and receive the plurality of temporally ordered datasets comprising reconstructed image data represented by a plurality of reconstructed images. The proposed imaging apparatus further allows a filter action on the reconstructed medical images. The process of using the raw detector data to create an image is called image reconstruction. The reconstructed image data can be from the data encoded in sinogram. In CT imaging, once the medical images have been reconstructed from the raw detector data, so that each pixel or voxel is assigned a specific value used to express CT density in a standardized fashion. The reconstructed image data can be also from the data encoded in other form of transitional images. For example, magnet resonance imaging, MRI, is an imaging process collects raw detector data and converts them to the transitional data represented in the k-space. K-space is a domain that represents the spatial frequencies composition and the phase information of imaging the region of interest. Each point in k-space corresponds to a specific combination of spatial frequency and phase information. The MRI reconstructs the image from the k-space data into a medical image. The proposed imaging apparatus allows a filter action on the reconstructed medical images further increases the flexibility in the application of the imaging apparatus. In addition, the proposed imaging apparatus may allow a filter action on the sinograms and the reconstructed medical images simultaneously.

In a possible embodiment of the imaging apparatus, the data-acquisition module is configured such, that that the spatial domain information comprises 3D spatial domain information. The 3D spatial domain information can be obtained from the imaging that represents the region of interest in the dimensions of length, width and depth. For example, the 3D spatial domain information can be obtained from CT scanning or the like, which are characterized by the tomographic imaging where the cross-sectional images from different layers of the region of interest are imaged and aggregated into a 3D image. The 3D spatial domain information can be obtained from MRI scanning or the like that use magnetic field gradients and electromagnetic pulses to spatially encode the intensity of a signal in the region of interest. The 3D spatial domain information can be obtained from PET scanning or the like that detect radiation rays from the annihilation of the particles to form a 3D medical image. Thus, in this case the time-resolved imaging can also be called as 4D imaging.

In a possible embodiment of the imaging apparatus, the data-acquisition module is configured such, that that the spatial domain information comprises 2D spatial domain information. The 2D spatial domain information can be obtained from the imaging that represents the region of interest in the dimension of length and width. The 2D spatial domain information can be obtained from traditional X-ray radiography or the like that uses X-ray to produce 2D images of regions of interest. The 2D spatial domain information can be obtained from optical microscopy or the like that produces 2D images of cells and tissues at the microscopic level using visible light. The 2D spatial domain imaging can be obtained from nuclear medicine planar scintigraphy that detects gamma rays emitted by radiotracers to create 2D images of physiological processes. The 2D spatial domain can be obtained from traditional 2D angiography that uses X-ray and a contrast agent injected into blood vessels to visualize their structure and flow. In these cases, the additional dimension offered by the time-resolved imaging is the time.

In a possible embodiment of the imaging apparatus, the data-acquisition module is configured to capture and receive the plurality of temporally ordered datasets in at least one of following predefined time periods: respiratory cycle; cardiac cycle; diffusion activity period; fluoroscopy imaging period; perfusion period. In the conventional 2D or 3D medical imaging processes, the motion of the regions of interest has a significant impact on the image quality. In some cases, even the respiratory activity of the patient will affect the image quality. The respiratory activity is substantially periodic. Time-resolved medical imaging takes advantage of this special feature. During a respiratory cycle or cardiac cycle, at least one predefined time point can be defined for taking a 2D or 3D image. For example, if only one predefined time point is defined in a respiratory cycle or cardiac cycle, at least for the next subsequent respiratory cycle or cardiac cycle, respectively, an image of the same predefined time point can be captured. If the pixel or voxel value differs too much from the surrounding pixel or voxel value from the same predefined time point but in different respiratory cycles, it can be assumed that it is noise. If more than one predefined time points are defined in a respiratory cycle or cardiac cycle, the proposed imaging apparatus can also use the information to denoise time-resolved medical images from the images taken from different predefined time points but in the same respiratory cycle, and alternatively, from the images taken from the different respiratory cycles or cardiac cycle, respectively. The more images are taken on the regions of interest, the more facilitative the noise filtering. The proposed imaging apparatus can also be used in other substantial periodic activities. As for the medical imaging processes that focused on medical activity at a specific time period, such as diffusion activity imaging, fluoroscopy, perfusion-imaging or the like, the proposed imaging apparatus can also be applied in such medical imaging processes. The pixel or voxel value can be also assumed that it is noise, when these pixel or voxel value differs too much from the surrounding pixel or voxel value from the scans that contains similar anatomy but are recoded at different predefined time points within one time period or time cycle.

In a possible embodiment of the imaging apparatus, the imaging apparatus comprises at least one of the following apparatuses: CT scanner; MRI scanner; PET scanner; X-ray machine. The CT scanner or the like, which are characterized by the tomographic imaging where the cross-sectional images from different layers of the region of interest are imaged and aggregated into a 3D image. The MRI scanner or the like uses magnetic field gradients and electromagnetic pulses to spatially encode the intensity of a signal in the region of interest. The PET scanner or the like detects radiation rays from the annihilation of the particles to form a 3D medical image. The X-ray machine or the like emits X-ray beams to penetrate the regions of interest to obtain the projection data used for reconstructing a medical image. The X-ray machine can be used to radiography processes, fluoroscopy systems, mammography, angiography and the like. The imaging apparatus proposed here enables cross-platform applications and improves the accessibility and flexibility of applications.

In a possible embodiment of the imaging apparatus, the at least one denoising module further comprises an optimization module that is configured to set/adjust the settable/adjustable bilateral filter by implementing an optimization algorithm, wherein the optimization algorithm allows that a parametrization of the settable/adjustable bilateral filter is set automatically. The setting/adjusting process of the settable/adjustable bilateral filter is computer-executable. The settable/adjustable bilateral filter may comprise at least one settable/adjustable bilateral filter layer. There might be a plurality of settable/adjustable bilateral filter layers established by the computer-executable denoising module, which each settable/adjustable bilateral filter layer may have different filter parameters according to different noise characteristic. The input settable/adjustable parameters of the settable/adjustable bilateral filter may be from low-SNR medical images acquired through the simulation of low-SNR images out of the existing images with high image quality. The optimization algorithm may involve a backpropagation algorithm. With the implementation of the backpropagation algorithm, the difference between the predicted image outputs and the actual images with high image quality is propagated backward through the settable/adjustable bilateral filter to optimize the parametrization. The settable/adjustable bilateral filter layer can be differentiable, which means the backpropagation algorithm can be used as a gradient estimation method to update the parameters through reducing the value of cost/loss function during the setting/adjusting of the bilateral filter. Therefore, the parametrization of the settable/adjustable bilateral filter is set automatically. In this process, the filter action on the sinograms and the reconstructed medical images can be conducted simultaneously. The setting/adjusting of the bilateral filter may be a self-supervised training. In self-supervised training process, the training parameters may not need to be labeled manually, which is more time-and cost-effective, and the training parameters may be not stuck in the local optimas.

In a possible embodiment of the imaging apparatus, the optimization module is further configured such, that the parametrization of the settable/adjustable bilateral filter includes two or three spatial parameters, an intensity range parameter and a time-intensity profile of those parameters. According to the features of the bilateral filters and the time-resolved medical imaging, the settable/adjustable parameters may only use two spatial parameters and one intensity range value and the time-intensity profile of those parameters. Alternatively, the settable/adjustable parameters may only use three spatial parameters and one intensity range value and the time-intensity profile of those parameters. Bilateral filters typically involve domain filters and range filters in medical imaging. A domain filter operates based on the spatial characteristics of an image. It refers to modify the pixel or voxel values of the data based on the location of the pixels or voxel values. A range filter selectively processes pixel or voxel values based on their intensity, in order to highlight or suppress specific ranges of brightness. When the settable/adjustable parameters of the bilateral filter may only use two spatial parameters and one intensity range value and the time-intensity profile of those parameters, the settable/ adjustable parameters may refer to the spatial kernel size widths with regard to the length dimension and width dimension of the image data used in an image generation process, the intensity range kernel size width and time-intensity profile of those parameters. When the settable/adjustable parameters of the at least one bilateral filter may only use three spatial parameters and one intensity range value and the time-intensity profile of those parameters, the settable/adjustable parameters may refer to the spatial kernel size widths with regard to the length dimension, width dimension and the depth dimension of the image data used in an image generation process, the intensity rang kernel size width and time-intensity profile of those parameters. The kernel may be a Gaussian kernel, which is used to apply Gaussian smoothing to the images by convolving the images with a Gaussian function. Gaussian kernel is essentially a 2D or 3D matrix where values, also called weights in the matrix, closer to the center of the matrix are higher, and values decrease symmetrically as they move away from the center. Compared to the other methods for denoising using convolutional neural networks or the like that uses thousands of parameters, the setting/adjusting process of the proposed method uses only few parameters, which can achieve similar filtering performance with low computational load. The method can be also explainable and produce no hallucinations since it is based on established filter operations that can be optimized and finely tuned for every specific noise characteristic.

In a possible embodiment of the method, an order of the plurality of temporally ordered datasets and theirs weighting used in the time-intensity profile are decided according to at least one of the following: temporal proximity; anatomical similarity; automated similarity metric. The temporally ordered datasets are not necessary ordered with sequential time order. The images are taken at different time points, can be ordered according to the temporal proximity of the time-resolved imaging, can be according to the anatomical similarity of the time-resolved imaging, or can be according to an automated similarity metric, such as structural similarity index, SSIM, mean squared error, MSE or the like. In different specific use scenarios, the weighting of the time-intensity profile may be different, therefore, the plurality of temporally ordered datasets are not necessary ordered with sequential time order. If the pixel or voxel value differs too much from the surrounding pixel or voxel values from the same points of the region of interest recorded with different temporally orders, it can be assumed that it is noise.

In a possible embodiment of the method, when the step of capturing and receiving the plurality of temporally ordered datasets is involving an ionizing radiation, an amount of deposited radiation dose that necessary to acquire a medical image is reduced. Ionizing radiation, such as X-ray, gamma ray or the like, used in medical imaging can cause stochastic effects in living tissue. The effective dose limit for an adult is limited to 20 milliSievert (mSv) in any single year. The effective dose assesses the potential for long-term effects after the radiation exposure that might occur in the further. The absorbed dose assesses the potential for biochemical changed in specific tissues after the radiation exposure, measured in milligray (mGy). Since the calculation of the effective dose is proportional to the absorbed dose to all organs of the body, the higher the absorbed dose, the more serious the radiation damage may occur to the body. Normally, in CT scanning, in order to allow for time-resolved image reconstruction with acceptable image quality, a comparably high radiation dose of 30-60 mGy is required. The proposed denoising method denoises the low-dose images, such that the denoised low-dose images having compatible performance with the high-dose images. Using the proposed denoising method, the noise reduction can be applied to reduce the dose necessary to acquire a medical image, which in the case of the medical imaging involving an ionizing radiation additional repeat imaging could be allowed to adapt the treatment plan.

In a possible embodiment of the method, the method operates with registration which. Registration brings a significant improvement, in particular the performance of the filters may be increased when it is combined with the registration. However, it goes without saying that the present method may also operate without registration. Registration in time-resolved medical imaging refers to the process of aligning two or more images into a common coordinate system. It preferably ensures that the anatomical or functional features in the images correspond spatially, even if the images were taken at different times, with different modalities, or from different viewpoints. Since the time-resolved medical imaging process takes the images in temporally order in at least one time period, the registration process is automatically finished during the imaging capturing process. When over time a region of interest no longer contains the same tissue, the information at these time points would be inherently excluded through the intensity threshold of the bilateral filter. This is important since deformable registration is a non-trivial task with a high computational load and its own margin of error.

Where appropriate, the above-mentioned configurations and developments can be combined implementations can be combined with each other as desired, as far as this is reasonable. Further possible configurations, developments and implementations of the invention also include combinations, which are not explicitly mentioned, of features which have been described previously or are described in the following with reference to the embodiments.

In the drawings, like, functionally equivalent and identically operating elements, features and components are provided with like reference signs in each case, unless stated otherwise.

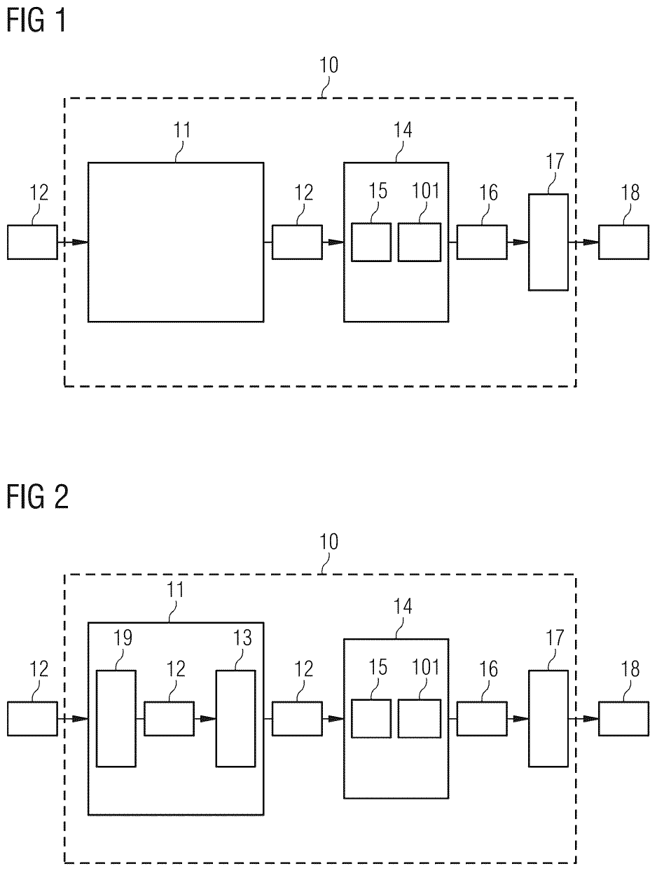

FIG. 1 illustrates a block diagram of an example of the image apparatus.

The image apparatus in FIG. 1 is denoted by reference numeral 10. The image apparatus 10 comprises a data-acquisition module 11, at least one denoising module 14 and an outputting module 17. The data-acquisition module 11 is configured to capture and receive a plurality of temporally ordered datasets 12 used in an image generation process for an entity in at least one predefined time period, such that the plurality of temporally ordered datasets 12 comprise spatial domain information and time domain information. The receiving of the plurality of temporally ordered datasets 12 may refer to store the captured datasets temporarily and wait for it to be processed later. The receiving of the plurality of temporally ordered datasets 12 may also refer to receive the datasets from external data sources. The at least one denoising module 14 is coupled to the data-receiving module 13 and comprises a settable/adjustable bilateral filter 15. The at least one denoising module 14 is configured to set/adjust filter parameters of the settable/adjustable bilateral filter 15 using the received spatial domain information and the time domain information, such to denoise the plurality of temporally ordered datasets 12 using a settable/adjustable bilateral filter 15 and to obtain a plurality of denoised temporally ordered datasets 16. The outputting module 17 is coupled to the at least one denoising module 14 and is configured to output at least one time-resolved medical image 18 for the entity using the plurality of denoised temporally ordered datasets 16.

FIG. 2 illustrates a block diagram of another example of the image apparatus. In FIG. 2, the data-receiving module 13 comprises an inputting module 19 for capturing plurality of temporally ordered datasets 12 and a data-receiving module for receiving the captured plurality of temporally ordered datasets 12. The rest part of the FIG. 2 is basically the same as illustrated in FIG. 1.

The entity to be imaged may refer to the regions of interest of a patient, a sick animal, a healthy person or animal providing comparative data through medical imaging for research or other purposes. The entity to be imaged may also refer to the non-living objects, such as imaging phantoms that designed to mimic human or animal anatomy or tissue properties.

The plurality of temporally ordered datasets 12 can comprise raw detector data described by a plurality of sinograms. Sinogram is used as a 2D transitional image between the raw detector data and the final medical image in some of the medical imaging processes. The proposed imaging apparatus allows a direct filter action on the sinogram. The proposed imaging apparatus can conduct filter function directly on the sinograms, which facilitates the later data processing step with less noises.

The plurality of temporally ordered datasets 12 can comprises reconstructed image data represented by a plurality of reconstructed images. The proposed imaging apparatus further allows a filter action on the reconstructed medical images. The process of using the raw detector data to create an image is called image reconstruction. The reconstructed image data can be from the data encoded in sinogram. The reconstructed image data can be also from the data encoded in other form of transitional images, such as k-space images or other more. The proposed imaging apparatus allows a filter action on the reconstructed medical images further increases the flexibility in the application of the imaging apparatus. In addition, the proposed imaging apparatus may allow a filter action on the sinograms and the reconstructed medical images simultaneously.

The plurality of temporally ordered datasets 12 comprise spatial domain information and time domain information, which the spatial domain information comprises 3D spatial domain information. The 3D spatial domain information can be obtained from the imaging that represents the region of interest in the dimensions of length, width and depth. The time is considered as the additional dimension of the medical imaging. Thus, in this case the time-resolved imaging can also be called as 4D imaging.

The plurality of temporally ordered datasets 12 comprise spatial domain information and time domain information, which the spatial domain information comprises 2D spatial domain information. The 2D spatial domain information can be obtained from the imaging that represents the region of interest in the dimension of length and width. In this case, the additional dimension offered by the time-resolved imaging is the time.

The plurality of temporally ordered datasets 12 are captured and received in at least one predefined time period, which the at least one predefined time period may be at least one respiratory cycle or cardiac cycle; at least one diffusion activity period; at least one fluoroscopy imaging period; at least one perfusion period. The time periods that can be represented by time cycles may be substantially periodic, such as the respiratory cycles or cardiac cycles, respectively. Time-resolved medical imaging takes advantage of this special feature. The proposed imaging apparatus 10 can also be used in other substantial periodic activities. If the pixel or voxel value differs too much from the surrounding pixel or voxel value from the same predefined time point but in different cycles, it can be assumed that it is noise. If more than one predefined time points are defined in a time cycle, the proposed imaging apparatus 10 can also use the information to denoise time-resolved medical images from the images taken from different predefined time points but in the same time cycle, and alternatively, from the images taken from the different time cycles. As for the medical imaging processes that focused on medical activity at a specific time period, such as diffusion activity imaging, fluoroscopy, perfusion-imaging or the like, the proposed imaging apparatus 10 can also be applied in such medical imaging processes. The pixel or voxel value can be also assumed that it is noise, when these pixel or voxel value differs too much from the surrounding pixel or voxel value from the scans that contains similar anatomy but are recoded at different predefined time points within one time period or time cycle.

The at least one denoising module 14 may further comprise an optimization module 101. The optimization module 101 is configured to set/adjust the settable/adjustable bilateral filter 15 by implementing an optimization algorithm, which the optimization algorithm allows that a parametrization of the settable/adjustable bilateral filter 15 is set automatically. The setting/adjusting process of the settable/adjustable bilateral filter 15 is computer-executable. The settable/adjustable bilateral filter 15 may comprise at least one settable/adjustable bilateral filter layer. There might be a plurality of settable/adjustable bilateral filter layers established by the computer-executable denoising module, which each settable/adjustable bilateral filter layer may have different filter parameters according to different noise characteristic. The input setting/adjusting parameters of the settable/adjustable bilateral filter 15 may be from low-SNR medical images acquired through the simulation of low-SNR images out of the existing images with high image quality. The optimization algorithm may involve a backpropagation algorithm. With the implementation of the backpropagation algorithm, the difference between the predicted image outputs and the actual images with high image quality is propagated backward through the settable/adjustable filter to optimize the parametrization. The settable/adjustable bilateral filter layer can be differentiable, which means the backpropagation algorithm can be used as a gradient estimation method to update the parameters through reducing the value of cost/loss function during the training of bilateral filter. Therefore, the parametrization of the settable/adjustable bilateral filter is set automatically. In this process, the filter action on the sinograms and the reconstructed medical images can be conducted simultaneously. The setting/adjusting of the settable/adjustable bilateral filter may be a self-supervised training. In self-supervised training process, the training parameters may not need to be labeled manually, which is more time-and cost-effective, and the training parameters may be not stuck in the local optimas.

The optimization module 101 is further configured such, that the parametrization of the settable/adjustable bilateral filter includes two or three spatial parameters, an intensity range parameter and a time-intensity profile of those parameters.

The imaging apparatus 10 may be CT scanner, MRI scanner, PET scanner, X-ray machine. Therefore, the imaging apparatus 10 proposed here enables cross-platform applications and improves the accessibility and flexibility of applications.

The outputting module 17 outputs at least one time-resolved image 18 for the entity using the plurality of denoised temporally ordered datasets 16. The at least one time-resolved image 18 can be received and displayed by at least one screen integrated into the imaging apparatus 10. The at least one time-resolved image 18 can be received and displayed by at least one screen with wires or wirelessly connected the imaging apparatus 10. The at least one time-resolved image 18 can be received and displayed in a cloud-based repository linked to the imaging apparatus 10 so that another person can download the at least one time-resolved image 18 from the cloud-based repository. The at least one time-resolved image 18 can be stored in at least one machine code, such that other person can obtain the at least one time-resolved image 18 by decoding the at least one machine code.

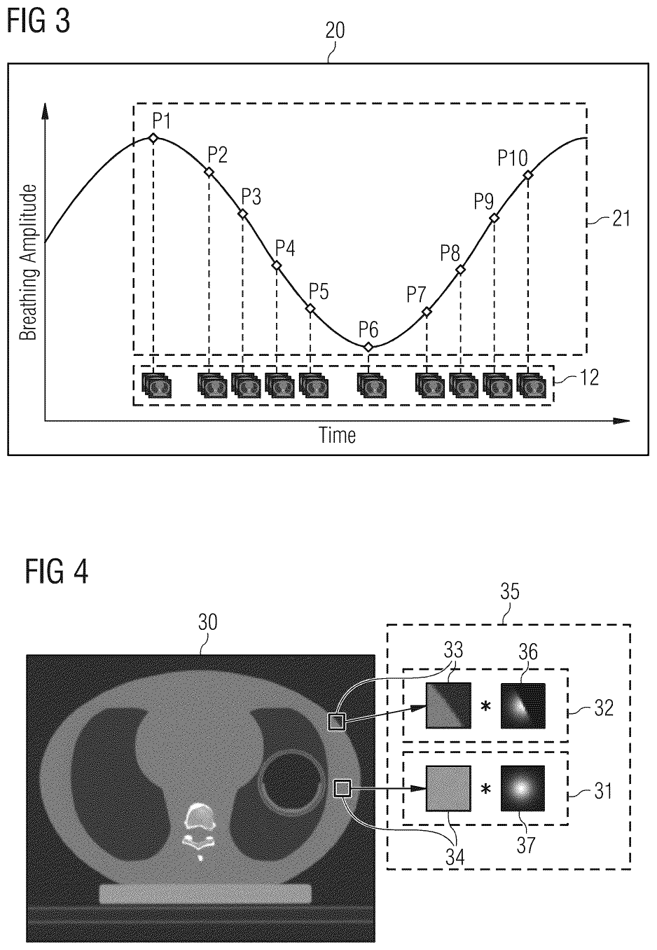

FIG. 3 illustrates a diagram of an example of a plurality of temporally ordered datasets captured within a time period. The diagram 20 represents that a plurality of temporally ordered datasets captured within a respiratory cycle 21. The vertical axis represents the breathing amplitude. The horizontal axis represents the time. At least ten time points P1, P2, . . . , P10 are predefined within the respiratory cycle 21. For each time point, a dataset is captured. After one respiratory cycle 21, at least ten datasets are captured and ordered temporally to obtain a plurality of temporally ordered datasets 12. Each dataset of the plurality of temporally ordered datasets 12 may represents a 3D volume, as illustrated in FIG. 2. The predefined time points within one time period might be more or less than ten time points.

For example, if only one predefined time point is defined in a respiratory cycle, at least for the next subsequent respiratory cycle, an image of the same predefined time point can be captured. If the pixel or voxel value differs too much from the surrounding pixel or voxel value from the same predefined time point but in different respiratory cycles, it can be assumed that it is noise. If more than one predefined time points are defined in a respiratory cycle, the proposed imaging apparatus can use the information to denoise time-resolved medical images from the images taken from different predefined time points but in the same respiratory cycle, and alternatively, from the images taken from the different respiratory cycles. Therefore, the more images are taken on the regions of interest, the more facilitative the noise filtering. The proposed imaging apparatus can also be used in other substantial periodic activities. As for the medical imaging processes that focused on medical activity at a specific time period, such as diffusion activity imaging, fluoroscopy, perfusion-imaging or the like, the proposed imaging apparatus can also be applied in such medical imaging processes. The pixel or voxel value can be also assumed that it is noise, when these pixel or voxel value differs too much from the surrounding pixel or voxel value from the scans that contains similar anatomy but are recoded at different predefined time points within one time period or time cycle.

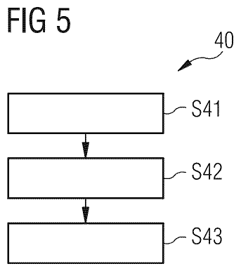

FIG. 4 illustrates a schematic diagram of an example of a filtering process for an exemplar image using a settable/adjustable bilateral filter.

In FIG. 4, a 2D reconstructed medical image 30 is taken as an example to present the filter process of one of the reconstructed medical images 30 in the plurality of temporally ordered reconstructed medical image using a settable/adjustable bilateral filter 35. The settable/adjustable bilateral filter 35 involves a domain filter 31 and a range filter 32 implementing Gaussian kernels. Gaussian kernel is essentially a 2D matrix in this example, where values, also called weights in the matrix, closer to the center of the matrix are higher, and values decrease symmetrically as they move away from the center. The domain filter 31 operates based on the spatial characteristics of an image. It refers to modify the intensity value of the pixel region 34 based on performing a convolution process with the normalized Gaussian spatial kernel 37 and the pixel region 34. Therefore, a denoised pixel region having the same imaging region of the pixel region 34 can be obtained. The pixel region 34 has been smoothed by the domain filter 31. This filter process reduces random fluctuations of the surrounding of the pixel region 34. In this example, the shape of the Gaussian spatial kernel 37 has two dimensions, so that the shape of the Gaussian spatial kernel 37 can be set/adjusted with regard to its length dimension and width dimension according to different noise characteristics. The range filter 32 processes pixel values based on their intensity, in order to highlight or suppress specific ranges of brightness. It refers to modify the intensity value of the pixel region 33 based on performing a convolution process with the normalized Gaussian range kernel 36 and the pixel region 33. Therefore, a denoised pixel region having the same imaging region of the pixel region 33 can be obtained. The pixel region 33 has been denoised by the range filter 32, however, the edge within the pixel region 33 has been preserved. The shape of the Gaussian range kernel 36 can also be set/adjusted according to different noise characteristics.

The settable/adjustable bilateral filter 35 is successful in smoothing images while preserving edges in reconstructed medical image 30. The example of FIG. 3 can be extended by preforming filtering process in 3D reconstructed medical image. In this scenario, the pixel region is replaced by a voxel region. The size of Gaussian spatial kernel 37 has three dimensions, so that the shape of the Gaussian spatial kernel 37 can be adjusted with regard to its length dimension, width dimension and depth dimension.

Each pixel or voxel of the reconstructed medical image might be performed such filtering process to conduct denoising, such that a denoised reconstructed medical image with high SNR can be obtained.

FIG. 5 illustrates a chart of an example of a method for time-resolved medical imaging.

The proposed method 40 comprises three steps: step S41, capturing and receiving a plurality of temporally ordered datasets 12 used in an image generation process for an entity in at least one predefined time period, such that the plurality of temporally ordered datasets 12 comprise spatial domain information and time domain information; step S42, setting/adjusting a settable/adjustable bilateral filter 15 using the spatial domain information and the time domain information of the plurality of temporally ordered datasets 12, such to denoise the plurality of temporally ordered datasets 12 using a settable/adjustable bilateral filter 15 and to obtain a plurality of denoised temporally ordered datasets 16; step S43, outputting at least one time-resolved medical image 18 for the entity using the plurality of denoised temporally ordered datasets 16.

Besides the description of FIG. 4, the filtering process of the plurality of temporally ordered datasets 12 further involves the time-intensity profile of the spatial parameters and the range parameter. An order of the plurality of temporally ordered datasets 12 and their weighting used in the time-intensity profile are decided according to at least one of the following: temporal proximity; anatomical similarity; automated similarity metric. The temporally ordered datasets are not necessary ordered with a sequential time order. The images are taken at different time points, can be ordered according to the temporal proximity of the time-resolved imaging, can be according to the anatomical similarity of the time-resolved imaging, or can be according to an automated similarity metric, such as structural similarity index, SSIM, mean squared error, MSE or the like. In different specific use scenarios, the weighting of the time-intensity profile may be different, therefore, the plurality of temporally ordered datasets are not necessary ordered with sequential time order. If the pixel or voxel value differs too much from the surrounding pixel or voxel values from the same points of the region of interest recorded with different temporally orders, it can be assumed that it is noise.

Compared to the other methods to training the bilateral filter using convolutional neural networks or the like that uses thousands of parameters to train the bilateral filters, the setting/adjusting process of the proposed method uses only few parameters, which can achieve similar filtering performance with low computational load. The method can be also explainable and produces no hallucinations since it is based on established filter operations that can be optimized and finely tuned for every specific noise characteristic. With the adoption of the time-intensity profile, when capturing the data used in an image generation process is involving an ionizing radiation and/or long acquisition time, an amount of deposited radiation dose and a prolonging acquisition time that necessary to acquire a medical image is reduced without compromising the image quality, such as contrast, temporal resolution and geometric accuracy. Furthermore, the method can be operated without registration.

Although the present invention has been described in the above by way of embodiments, it is not limited thereto, but rather can be modified in a wide range of ways. In particular, the invention can be changed or modified in various ways without deviating from the core of the invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. An imaging apparatus, the imaging apparatus comprising:
a data-acquisition module configured to capture and receive a plurality of temporally ordered datasets used in an image generation process for an entity in at least one predefined time period, wherein the plurality of temporally ordered datasets comprise spatial domain information and time domain information;
at least one denoising module coupled to the data-acquisition module, the at least one denoising module including a bilateral filter, the bilateral filter being at least one of settable or adjustable, wherein the at least one denoising module is configured to at least one of set or adjust filter parameters of the bilateral filter using the received spatial domain information and the time domain information to denoise the plurality of temporally ordered datasets using the bilateral filter and to obtain a plurality of denoised temporally ordered datasets; and
an outputting module configured to output at least one time-resolved medical image for the entity using the plurality of denoised temporally ordered datasets.

2. The imaging apparatus of claim 1, wherein the data-acquisition module is configured to capture and receive the plurality of temporally ordered datasets, the plurality of temporally ordered datasets including raw detector data described by a plurality of sinograms.

3. The imaging apparatus of claim 1, wherein the data-acquisition module is configured to capture and receive the plurality of temporally ordered datasets, the plurality of temporally ordered datasets including reconstructed image data represented by a plurality of reconstructed images.

4. The imaging apparatus of claim 1, wherein the spatial domain information comprises three-dimensional spatial domain information.

5. The imaging apparatus of claim 1, wherein the spatial domain information comprises two-dimensional spatial domain information.

6. The imaging apparatus of claim 1, wherein the data-acquisition module is configured to capture and receive the plurality of temporally ordered datasets in at least one of:
a respiratory cycle;
a cardiac cycle;
a diffusion activity period;
a fluoroscopy imaging period; or
a perfusion period.

7. The imaging apparatus of claim 1, further comprising at least one of:
a computed tomography (CT) scanner;
a magnet resonance imaging (MRI) scanner;
a positron emission tomography (PET) scanner; or
an X-ray machine.

8. The imaging apparatus of claim 1, wherein the at least one denoising module further comprises an optimization module configured to at least one of set or adjust the bilateral filter by implementing an optimization algorithm, wherein the optimization algorithm permits a parametrization of the bilateral filter to be set automatically.

9. The imaging apparatus of claim 8, wherein the parametrization of the bilateral filter includes two or three spatial parameters, an intensity range parameter and a time-intensity profile of the two or three spatial parameters and the intensity range parameter.

10. A method for time-resolved medical imaging, the method comprising:
capturing and receiving a plurality of temporally ordered datasets used in an image generation process for an entity in at least one predefined time period, wherein the plurality of temporally ordered datasets comprise spatial domain information and time domain information;
at least one of setting or adjusting filter parameters of a bilateral filter using the received spatial domain information and the time domain information to denoise the plurality of temporally ordered datasets using the bilateral filter and to obtain a plurality of denoised temporally ordered datasets; and
outputting at least one time-resolved medical image for the entity using the plurality of denoised temporally ordered datasets.

11. The method of claim 10, wherein an order of the plurality of temporally ordered datasets and associated weighting used in a time-intensity profile are decided according to at least one of:
a temporal proximity;
an anatomical similarity; or
an automated similarity metric.

12. The method of claim 10, wherein when the capturing and receiving the plurality of temporally ordered datasets is associated with an ionizing radiation, an amount of deposited radiation dose to acquire a medical image is reduced.

13. The method of claim 10, wherein the method operates with registration.

14. A non-transitory computer program product including program sections, when executed by a computer, cause an imaging apparatus to perform the method of claim 10.

15. A non-transitory computer-readable storage medium storing program sections, when executed by a computer, cause an imaging apparatus to perform the method of claim 10.

16. The imaging apparatus of claim 2, wherein the data-acquisition module is configured to capture and receive the plurality of temporally ordered datasets in at least one of:
a respiratory cycle;
a cardiac cycle;
a diffusion activity period;
a fluoroscopy imaging period; or
a perfusion period.

17. The imaging apparatus of claim 16, further comprising at least one of:
a computed tomography (CT) scanner;
a magnet resonance imaging (MRI) scanner;
a positron emission tomography (PET) scanner; or
an X-ray machine.

18. The imaging apparatus of claim 17, wherein the at least one denoising module further comprises an optimization module configured to at least one of set or adjust the bilateral filter by implementing an optimization algorithm, wherein the optimization algorithm permits a parametrization of the bilateral filter to be set automatically.

19. The imaging apparatus of claim 18, wherein the parametrization of the bilateral filter includes two or three spatial parameters, an intensity range parameter and a time-intensity profile of the two or three spatial parameters and the intensity range parameter.

20. The imaging apparatus of claim 2, wherein the data-acquisition module is configured to capture and receive the plurality of temporally ordered datasets, the plurality of temporally ordered datasets including reconstructed image data represented by a plurality of reconstructed images.
